(19) United States
(12) Patent Application Publication
(10) Pub. No.: US 2025/0049662 A1
(43) Pub. Date: Feb. 13, 2025

(54) AQUEOUS COSMETIC COMPOSITION COMPRISING VANILLIN OR ONE OF ITS DERIVATIVES, A SPECIFIC SEQUESTERING AGENT, AND POSSIBLY AT LEAST ONE ADDITIONAL ANTIMICROBIAL

(71) Applicant: L'OREAL, Paris (FR)

(72) Inventors: Nathalie GUILLIER, Chevilly la Rue (FR); Géraldine LEREBOUR, Chevilly la Rue (FR)

(73) Assignee: L'OREAL, Paris (FR)

(21) Appl. No.: 18/719,945

(22) PCT Filed: Dec. 21, 2022
(86) PCT No.: PCT/EP2022/087264

§ 371 Date: Jun. 14, 2024

(30) Foreign Application Priority Data
Dec. 21, 2021 (FR) FR2114134

(51) Int. Cl. A61K 8/34; A61K 8/44; A61Q 19/00

(52) U.S. Cl. CPC A61K 8/347; A61K 8/44; A61Q 19/00; A61K 2800/524

(57) ABSTRACT

The present invention relates to a composition, in particular cosmetic, comprising water and:
i) at least one (C₁-C₆)(alkyl)vanillin, one of the organic or mineral basic salts thereof, or one of the solvates thereof such as hydrates;
ii) at least one sequestering agent selected from the salts of formula (I) as well as the solvates thereof such as hydrates, and ethylene diamine disuccinic acid and the salts thereof, and
iii) optionally at least one additional antimicrobial, preferably selected from C₂ to C₆ alkanols, C₂ to C₁₀ polyols, surfactants in particular non-ionic and mixtures thereof.

The present invention relates to a composition, in particular cosmetic, comprising water and:
i) at least one (C₁-C₆)(alkyl)vanillin, one of the organic or mineral basic salts thereof, or one of the solvates thereof such as hydrates, preferably vanillin,
ii) at least one sequestering agent selected from the salts of formula (I) such as defined hereinafter, and ethylene diamine disuccinic acid and the salts thereof, and
iii) optionally at least one additional antimicrobial, preferably selected from C₂ to C₆ alkanols, C₂ to C₁₀ polyols, non-ionic surfactants and mixtures thereof.

Microorganisms can survive and multiply in products containing water, whether they are cosmetic, pharmaceutical or food products. Preservatives are generally added to industrial preparations intended to be stored or preserved to prevent microbial growth overtime. Microbial contamination can occur during the manufacture of industrial products, even when initial ingredients placed in said products are “clean”, i.e., devoid of contaminant microorganisms. Water for example, which is omnipresent in most cosmetic, pharmaceutical or food products, must be free from contaminant microorganisms. The other ingredients must also undergo screening for the presence of contaminant microorganisms.

Cleanliness during the manufacture of these industrial products, the processing of content and filling of containers must be monitored meticulously. Despite these precautions, the microbial integrity of the products may require the presence of one or more preservative agents compatible with the product and the stability of the composition. The preservative agents must not allow the growth or viability of the contaminant microorganisms.

Although restrictive production in a sterile environment may be industrially feasible, maintaining sterility over repeated uses represents a problem in its own right, in particular when the products can be touched by fingers and/or instruments such as applicators, and/or the container thereof can be open and closed, as all these handlings are sources of contamination.

The use of preservative agents is thus customary to reduce the microbial contamination added by consumers under normal conditions of use. As a general rule, pathogenic microorganisms must be absent from all marketed products, particularly cosmetic.

The compounds that can be used for the microbial protection of particularly cosmetic compositions must moreover be stable, in order to ensure the efficacy thereof overtime and not exhibit solubility, odor and/or compatibility characteristics that render them difficult to use. It is moreover desirable to diversify the compounds that can be used in antimicrobial protection so as not to generate resistances.

Therefore, there is a need for novel ingredients or novel ingredient mixtures which are effective in the antimicrobial protection of particularly cosmetic compositions, which are stable, easy to formulate and which generate no odor or color after incorporation in said compositions, particularly after several months of storage at a temperature greater than or equal to 25° C., particularly between 37° C. and 45° C.

Furthermore, there is also a need to formulate compositions which comprise several antimicrobials, which remain stable over time, and which particularly do not change appearance and/or color over time, even after several weeks or several months of storage at a temperature greater than or equal to 25° C., particularly between 37° C. and 45° C. It is particularly important that the composition does not change color over time especially for light cosmetic compositions such as very light or brightening foundation creams, care creams (that are often white) and other compositions applied on the skin and hair. Indeed, for consumers, the change of color may indicate that the active ingredients have changed and that the product has become unfit for consumption. The use of vanillin in compositions is problematic as it is an ingredient that can change color over time, to dark brown, more or less quickly depending on the environment thereof particularly the temperature, oxygen, and/or light.

The present invention meets these needs.

Surprisingly, the Applicant identified mixtures of compounds i) and ii) having a marked antimicrobial efficacy. These specific mixtures remain stable even after several weeks, or several months, at ambient temperature or even at temperatures greater than 25° C., particularly between 37 and 45° C.

Moreover, such mixtures do not exhibit an unpleasant odor over time. Furthermore, no noteworthy change in odor over time was observed even after several weeks of storage.

The present invention thus relates to a composition, in particular cosmetic, comprising water and:
i) at least one (C₁-C₆)(alkyl)vanillin, one of the organic or mineral base salts thereof, or one of the solvates thereof such as hydrates;
ii) at least one sequestering agent selected from:
   a) the salts of the following formula (I), as well as the solvates thereof such as hydrates:

[Chem 1]

[⁻O—C(O)—CH₂]₂N—CH[C(O)O⁻]—(CH₂)₂—C(O)O—, nM⁺ (I)
Formula (I) wherein:
   M⁺, identical or different, is selected from H⁺, alkali metal cations, alkaline earth metal cations and ammonium ions, and
   n is an integer such that the number of M+ ensures the electroneutrality of the molecule, preferably between 2 and 4, more specifically equal to 2 or 4, and
b) ethylene diamine disuccinic acid as well as the optical isomers thereof, and organic or mineral base salts thereof, preferably mineral in particular M⁺ OH⁻with M⁺ such as defined hereinabove, and
c) the mixture of a) and b),
iii) optionally at least one additional antimicrobial, preferably selected from C₂ to C₆ alkanols, C₂ to C₁₀ polyols, surfactants in particular non-ionic and mixtures thereof.

Preferably the sequestering agent(s) are selected from a).

The association of i), ii) and optionally iii) corresponds to the antimicrobial mixture.

The invention also relates to a non-therapeutic method for the cosmetic treatment of keratin materials, comprising application of a composition according to the invention on said keratin materials. The invention also relates to a non-therapeutic cosmetic method for the care and/or makeup and/or cleansing of keratin materials, comprising application of a composition according to the invention on said keratin materials.

The invention also relates to a method for preserving a composition C1 comprising a physiologically acceptable medium comprising water, in particular a cosmetic or dermatological composition C1, which comprises the incorporation of the compounds i) and ii) and optionally iii) according to the invention in said composition C1. The term “physiologically acceptable medium” denotes a medium compatible with the skin, mucosa and/or appendages (including keratin fibers, particularly human, such as hair).

The invention also relates to the use of a composition according to the invention as an antimicrobial agent, in particular against bacterial strains (for example Enterococcus faecalis), yeasts (for example Candida albicans) and/or molds (for example Aspergillus brasiliensis).

### i) Vanillin and Alkoxylated Derivative

The composition according to the invention comprises at least one (C₁-C₆)(alkyl)vanillin or one of the organic or mineral basic salts thereof, or one of the solvates thereof such as hydrates.

The term “(C₁-C₆)(alkyl)vanillin” denotes vanillin, i.e., 3-methoxy-4-hydroxybenzaldehyde and derivatives thereof including a (C₂-C₆)alkyl group in the place of methyl of the methoxy at position 3 of 4-hydroxybenzaldehyde.

More specifically, (C₁-C₆)(alkyl)vanillin according to the invention has the following formula (II):

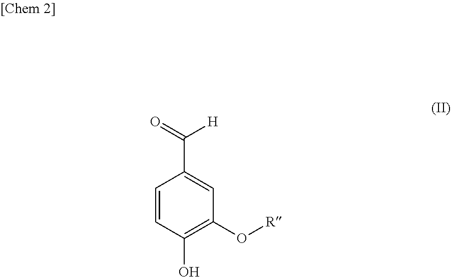

Formula (II) wherein R″ represents a linear or branched (C₁-C₆)alkyl group, preferably a (C₁-C₄)alkyl group such as methyl or ethyl, more preferably methyl.

A “(C₁-C₆)alkyl group” is a linear or branched, saturated C₁-C₆ group, preferably C₁-C₄ ((C₁-C₄)alkyl group) such as methyl or ethyl.

(C₁-C₆)(alkyl)vanillin, the organic or mineral basic salts thereof, or solvates thereof such as hydrates, can be of natural or synthetic origin. According to an embodiment of the invention, (C₁-C₆)(alkyl)vanillin, the organic or mineral basic salts thereof, or solvates thereof such as hydrates, is/are in organic or mineral basic salt form, such as alkali metal salts (Na⁺, K⁺), alkaline earth metal salts (Mg²⁺, Ca²⁺) and ammonium ions. It/they can be natural or synthetic origin.

The composition according to the invention preferably comprises a quantity of (C₁-C₆)(alkyl)vanillin, the organic or mineral basic salts thereof, or solvates thereof such as hydrates, between 0.01% and 2% by weight with respect to the total weight of the composition, preferably between 0.05% and 1% by weight, and even more preferably between 0.1% and 0.5% by weight with respect to the total weight of the composition.

### ii) Sequestering Agent

The composition according to the invention also comprises at least one sequestering agent selected from a) the salts of formula (I) such as defined hereinabove, b) ethylene diamine disuccinic acid and c) the mixture a)+b) thereof such as defined hereinabove.

According to a particular embodiment of the invention the sequestering agent(s) are a) of formula (I) such as defined hereinabove, wherein M⁺, identical or different, is selected from alkali metal cation, alkaline earth metal cations and ammonium ions.

When M⁺, identical or different, is selected from alkali metal cation and ammonium ions, then n is equal to 4.

When M⁺, identical or different, is selected from alkaline earth metal cations, then n is equal to 2.

The term “alkali metal cations” particularly denotes the ions Na⁺ and K⁺.

The term “alkaline earth metal cations” particularly denotes the ions Mg²⁺ and Ca²⁺.

Preferably, the cations M⁺ are identical.

Preferably, M⁺ is selected from alkali metal cations.

Preferably, M⁺ is Na⁺.

Preferably, the sequestering agent has the following formula (I′), as well as the optical isomers thereof, and the solvates thereof such as hydrates:

[Chem 3]

[⁻O—C(O)—CH₂]₂N—CH(COO—)—(CH₂)₂—C(O)O⁻, 4Na⁺ (I′)

This compound having formula (I′) is tetrasodium glutamate diacetate (CAS: 51981-21-6). This compound is particularly marketed by Akzo Nobel under the name Dissolvine GL-47-S.

According to another embodiment, the sequestering agent is selected from b) ethylene diamine disuccinic acid (EDDS) and the optical isomers thereof, the basic salts thereof such as defined hereinabove and the solvates thereof such as hydrates. The ethylene diamine disuccinic acid is the compound of formula (III):

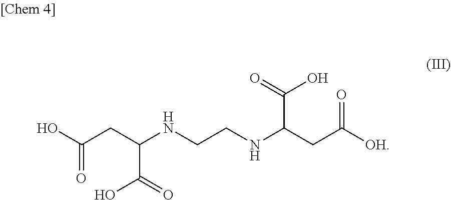 [SMILES: O=C(O)CC(NCCNC(CC(=O)O)C(=O)O)C(=O)O]

Preferably, the ethylene diamine disuccinic acid salt is selected from alkali metal salts, such as potassium and sodium salts, ammonium salts, and amine salts. The alkali metal salts of ethylenediamine disuccinic acid are more specifically preferred.

Preferably, the ethylene diamine disuccinic acid salt used according to the invention is trisodium ethylene diamine disuccinate. Such a compound is for example that marketed under the trade name Natrlquest® E30 by Innospec Active Chemicals (dispersion of the compound at 37% by weight of active substance in water), or that marketed under the trade name Octaquest E30® by Octel Performance Chemicals.
c) according to another embodiment a mixture a)+b).

According to another embodiment, in addition to the sequestering agents a), b) and the mixture thereof c) another sequestering agent can be added to a), b) or c) which is the sequestering agent in particular selected from ethylene diamine tetraacetic acid (EDTA) and the salts thereof. Preferably, the EDTA salt is selected from alkali metal salts, such as sodium salts, particularly the disodium salt of ethylene diamine tetraacetic acid (disodium EDTA).

The composition according to the invention preferably comprises a quantity of sequestering agent having formula (I) or (I′), or of EDDS or one of the salts thereof, comprised between 0.01% to 1% by weight with respect to the total weight of the composition, preferably ranging from 0.05% to 0.7% by weight, and more preferably ranging from 0.1% to 0.5% by weight.

### III) Additional Antimicrobial

The composition according to the invention can comprise at least one additional microbial A. Thus, said additional antimicrobial A is not (C₁-C₆)(alkyl)vanillin or its salts and/or solvates i), and is likewise not the sequestering agent ii) having formula (I) as defined above.

The term “antimicrobial agent” or “antimicrobial” or “preservative” or “preservative agent” denotes any cosmetically or pharmaceutically acceptable compound, which helps prevent the growth of microorganisms capable of occurring in particularly cosmetic or pharmaceutical compositions, from the design thereof, through the storage, until the conventional use thereof by consumers. By way of preservatives, mention can particularly be made of the preservatives described in Cosmetics, Kirk-Othmer Encyclopedia of Chemical Technology. John Wiley & Sons, Inc. Martin M. Rieger; 5.2 preservatives & table 3, 04/12/2000 https://doi.org/10.1002/0471238961.0315191318090507.a01 or https://library.essentialwholesale.com/alcohol/.

Preferably, the additional antimicrobial A can be selected from sodium dehydroacetate; benzene carboxylic acids optionally substituted on the phenyl group by one or more groups selected from hydroxy, (C₁-C₁₀)alkyl and (C₁-C₁₀)alkylacarbonyl, as well as the basic salts thereof particularly of alkali and alkaline earth metals, preferably benzoic acid or sodium benzoate; hydroxybenzoic acid esters optionally substituted on the phenyl group by one or more groups selected from (C₁-C₁₀)alkyl such as parabens particularly methylparaben, ethylparaben, propyl paraben or butylparaben; potassium sorbate; salicylic acid optionally substituted by a (C₁-C₈)alkylcarbonyl group, preferably salicylic acid; chlorhexidine digluconate, chlorphenesin, polyaminopropyl biguanide, benzyl alcohol, myrtrimonium bromide, LAE (Ethyl Lauroyl Arginate HCl), phenoxyethanol, octopirox, behentrimonium chloride, cetrimonium chloride, C₂ to C₆ alkanols, C₂ to C₁₀ polyols, pyridines substituted at position 3 by an amide group RR′N—C(O)— and solvates thereof such as hydrates, where R and R′, identical or different, represent a hydrogen atom, a (C₁-C₆)alkyl group such as methyl or ethyl, (C₂-C₆)alkenyl such as allyl, (hetero)aryl(C₁-C₄)alkyl such as benzyl or pycolyl, surfactants in particular non-ionic and mixtures thereof.

Particularly preferably, the additional antimicrobial A selected from C₂ to C₆ alkanols, C₂ to C₁₀ polyols, surfactants in particular non-ionic and mixtures thereof.

Preferably, the C₂ to C₆ alkanol is a C₂ to C₆ aliphatic monoalcohol. Preferably, said aliphatic monoalcohol comprises from 2 to 4 carbon atoms.

The term “aliphatic monoalcohol” denotes any saturated, linear or branched alkane compound having a single hydroxyl (OH) group. The aliphatic monoalcohol(s) present in the compositions according to the invention can be selected from ethanol, propanol, butanol, isopropanol, isobutanol and mixtures thereof. More specifically, ethanol will be selected.

The term “polyol” denotes any linear or branched saturated alkane including 2 to 10 carbon atoms substituted by at least 2 hydroxyl functions and optionally interrupted by one or more heteroatoms such as O, N, S particularly O, preferably from 2 to 4 hydroxyl groups. Preferably, the polyol comprises from 2 to 6 carbon atoms.

Preferably, the polyol(s) having from 2 to 10 carbon atoms are selected from glycerol, diglycerol, propyleneglycol, isoprene glycol, dipropyleneglycol, butylene glycol, hexylene glycol, 1,2-propanediol, 1,3-propanediol, pentylene glycol, heptane diols, caprylyl glycol and mixtures thereof, more preferably glycerol.

According to one embodiment the additional microbial A is a surfactant.

The term “surfactant” denotes a “surface agent” or “surfactant” that is a compound able to modify the surface tension between two surfaces, surfactants are amphiphilic molecules, i.e. that have two parts of different polarity, one is lipophilic and apolar and the other is hydrophilic and polar. The surfactant(s) can be cationic, anionic, non-ionic, or zwitterionic, preferably non-ionic.

Preferably the surfactant(s) are selected from C₆-C₂₀ hydrocarbon chain compounds having formula (IV) and the organic or mineral acid or basic salts thereof, the optical isomers thereof, and the solvates thereof such as hydrates:

[Chem 5]

R₁—C(O)—NH—CH(R₂)—C(O)—OR₃ (IV)
wherein:
   R₁ represents a (C₆-C₂₀)alkyl group;
   R₂ represents a hydrogen atom or a (C₁-C₆)alkyl group optionally substituted by a guanidine group optionally substituted by one or more (C₁-C₄)alkyl groups; and
   R₃ represents a hydrogen atom or a (C₁-C₆)alkyl group.

The term “organic or mineral acid salt” more specifically denotes salts selected from a salt derived from i) hydrochloric acid HCl, ii) hydrobromic acid HBr, iii) sulfuric acid H₂SO₄, iv) alkylsulfonic acids: Alk-S(O)₂OH such as methylsulfonic acid and ethylsulfonic acid; v) arylsulfonic acids: Ar—S(O)₂OH such as benzene sulfonic acid and toluene sulfonic acid; vi) citric acid; vii) succinic acid; viii) tartaric acid; ix) lactic acid, x) alkoxysulfinic acids: Alk-O—S(O)OH such as methoxysulfinic acid and ethoxysulfinic acid; xi) aryloxysulfinic acids such as tolueneoxysulfinic acid and phenoxysulfinic acid; xii) phosphoric acid H₃PO₄; xiii) acetic acid CH₃C(O)OH; and xiv) triflic acid CF₃SO₃H and xv) tetrafluoroboric acid HBF₄.

The term “organic or mineral basic salt” denotes the salts of bases or alkaline agents as defined hereinafter such as alkali metal hydroxides like soda, potash, ammonia, amines or alkanolamines.

A “(C₆-C₂₀)alkyl group” is a linear or branched, saturated C₁-C₂₀ hydrocarbon radical, preferably C₁-C₆ ((C₁-C₆)alkyl group), more preferably C₁-C₄ ((C₁-C₄)alkyl group) such as methyl or ethyl.

Preferably, in formula (IV) such as defined hereinabove, R₁ represents a linear or branched, preferably linear, (C₆-C₂₀)alkyl group, more specifically R1 represents a linear or branched, preferably linear, (C₈-C₁₂)alkyl group.

Preferably, in formula (IV) such as defined hereinabove, R₂ represents a hydrogen atom. Alternatively, preferably, in formula (IV), R₂ represents a (C₁-C₆)alkyl group, preferably substituted by a guanidine group optionally substituted by one or more (C₁-C₄)alkyl groups. According to this embodiment, R₂ is substituted by a guanidine group —NH—C(═NH)—NH₂.

Preferably, in formula (IV) such as defined hereinabove, R₃ represents a hydrogen atom. Alternatively, preferably, in formula (IV), R₃ represents a (C₁-C₆)alkyl group.

Preferably, the compound having formula (IV) is selected from laurylethyl arginate and N-octanoyl glycine (also known as capryloyl glycine), preferably N-octanoyl glycine.

Preferably, the additional antimicrobial is selected from ethanol, pentylene glycol, 1,2-propanediol, 1,3-propanediol, capryloyl glycine and mixtures thereof.

Preferably, the additional antimicrobial agent is at least capryloyl glycine.

The composition according to the invention optionally comprises at least one C₂ to C₆ alkanol and/or C₂ to C₁₀ polyol at a content ranging from 1 to 20% by weight with respect to the total weight of the composition, preferably from 1.5% to 13% by weight, and more preferably from 2.0% to 8.0% by weight.

The composition according to the invention optionally comprises at least one non-ionic surfactant having formula (IV), preferably capryloyl glycine, at a content ranging from 0.01% to 3% by weight with respect to the total weight of the composition, preferably from 0.05% to 1.5% by weight, and more preferably from 0.2% to 0.8% by weight.

### pH of the Composition

The pH of the composition according to the invention is generally between 3 and 10 approximately, preferably between 3 and 8 approximately, preferably between 4 and 6 approximately, and preferably between 4 and 5.5, preferably between 4.2 and 5. Preferably, it is approximately 4.6.

It can be adjusted to the desired value by means of acidifying or alkaline agents routinely used for the treatment of keratin materials or using conventional buffer systems.

Of the acidifying agents, mention can be made, by way of example, of mineral or organic acids as defined above particularly those i) to xv) of “organic or mineral acid salt”. In particular, the acidifying agent is selected from hydrochloric acid, orthophosphoric acid, sulfuric acid, carboxylic acids such as acetic acid, tartaric acid, citric acid, lactic acid and sulfonic acids.

The term “alkaline agents” or “base” denotes an agent for including the pH of the composition wherein it is located. The alkalinizing agent is a Bronsted, Lowry or Lewis base. It can be mineral or organic. Specifically, said agent is selected from a) ammonia, b) (bi)carbonate, c) alkanolamines such as mono-, di- and triethanolamines as well as their derivatives d) oxyethylenated and/or oxypropylenated ethylenediamines, e) organic amines, f) mineral or organic hydroxides, g) alkali metal silicates such as sodium metasilicates, h) preferably basic amino acids such as arginine, lysine, ornithine, citrulline and histidine, and i) compounds having the following formula (F):

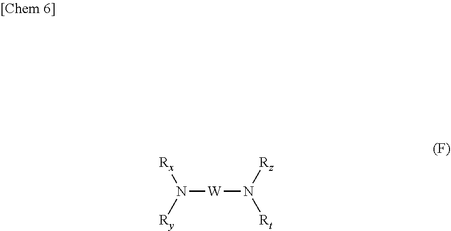

Formula (F) wherein:
W is a divalent C₁-C₆ alkylene radical optionally substituted by one or more hydroxyl groups of a C₁-C₆ alkyl radical, and/or optionally interrupted by one or more heteroatoms such as O, or NRᵤ;
Rₓ, R_{y}, R_{z} and Rₜ, identical or different, represent a hydrogen atom, a C₁-C₆ alkyl or C₁-C₆ hydroxyalkyl, C₁-C₆ aminoalkyl radical.

Mention can be made by way of example of amines having formula (F) of 1,3 diaminopropane, 1,3 diamino 2-propanol, spermine, spermidine.

The term “alkanolamine” denotes an organic amine comprising a primary, secondary or tertiary amine function, and one or more linear or branched C₁—C alkyl groups carrying one or more hydroxyl radicals.

Of the mineral or organic hydroxides, mention can be made of those selected from a) alkali metal hydroxides, b) alkaline earth metal hydroxides, such as sodium or potassium hydroxides, c) transition metal hydroxides, d) lanthanide or actinide hydroxides, quaternary ammonium hydroxides and guanidinium hydroxide, mineral or organic hydroxides a) and b) being preferred.

Of the bases or alkaline agents, mention can be made, by way of example, of the alkaline agents as described hereinafter and particularly of natural alkaline agents, in particular preferably basic amino acids such as arginine.

### iv) Fats

The composition further comprises optionally one or more fats.

The term “fat” denotes an organic compound insoluble in water at regular ambient temperature (25° C.) and at atmospheric pressure (760 mm of Hg) (solubility less than 5% and preferably 1% and even more preferably 0.1%). They have in the structure thereof at least one hydrocarbon chain including at least 6 carbon atoms or a chain of at least two siloxane groups. The fats are generally soluble in organic solvents under the same temperature and pressure conditions, such as for example chloroform, ethanol, benzene, petrolatum oil or decamethyl cyclopentasiloxane.

The fat(s) according to the invention are of natural or synthetic origin, preferably natural, more preferably of plant origin. These fats are preferably neither polyoxyethylenated nor polyglycerolated. They are different from fatty acids as salified fatty acids form soaps generally soluble in aqueous medium.

According to a specific embodiment of the invention, the composition comprises one or more fats non-liquid at 25° C. and at atmospheric pressure.

### Wax(es)

According to a specific embodiment, the composition according to the invention comprises one or more waxes.

The term “wax” according to the present invention denotes a lipophilic compound, solid at ambient temperature (25° C.), having a reversible solid/liquid change of state and a melting point greater than or equal to 30° C. of up to 200° C. and particularly up to 120° C.

In particular, the wax(es) suitable for the invention can have a melting point greater than or equal to 45° C., and in particular greater than or equal to 55° C. The composition according to the invention preferably comprises a wax content ranging from 3% to 20% by weight with respect to the total weight of the composition, in particular from 5 to 15%, more specifically from 6 to 15%.

According to a specific form of the invention, the composition according to the invention is solid, in particular anhydrous. It can then be found in stick form, polyethylene micro-waxes in the form of crystallites having a form factor at least equal to 2 having a melting point ranging from 70 to 110° C. and preferably 70 to 100° C. will be used, in order to reduce or even suppress the presence of strata in the solid composition.

### Pasty Compound(s)

According to a specific embodiment, the composition according to the invention comprises one or more pasty compounds.

According to the present invention, the term “pasty compound” denotes a lipophilic fat compound having a reversible solid/liquid change of state, having in the solid state, an anisotropic crystalline organization, and comprising at a temperature of 23° C. a liquid fraction and a solid fraction.

According to a specific embodiment of the invention, the composition comprises one or more fats liquid at 25° C. and at atmospheric pressure, particularly hydrocarbon fats.

Preferably, the composition according to the invention comprises one or more liquid hydrocarbon fats which are particularly selected from hydrocarbons and C₆-C₁₆ or with more than 16 carbon atoms up to 60 carbon atoms, preferably between C₆ and C₁₆, and in particular alkanes, oils of animal origin, oils of plant origin, glycerides or fluorinated oils of synthetic origin, fatty alcohols, fatty acid and/or fatty alcohol esters, silicones.

In particular, the liquid fat(s) are selected from non-silicone oils.

It is recalled that, according to the invention, the alcohols, esters and fatty acids have more specifically one or more linear or branched, saturated or unsaturated hydrocarbon group(s), comprising 6 to 60 carbon atoms, optionally substituted, in particular by one or more hydroxyl group(s) OH (in particular from 1 to 4 hydroxyl group(s)). If they are unsaturated, these compounds can comprise one to three unsaturation(s), preferably of 1 to 3 carbon-carbon bonds, optionally conjugated.

As regards C₆-C₁₆ alkanes, the latter are linear or branched, optionally cyclic, preferably the fat(s) iv) according to the invention are selected from linear or branched C₈-C₁₄, more preferably C₉-C₁₃ even more preferably C₉-C₁₂ alkanes. Byway of example, mention can be made of hexane, undecane, dodecane, tridecane, isoparaffins such as isohexadecane, isodecane or isododecane. The linear or branched hydrocarbons having more than 16 carbon atoms, can be selected from paraffin oils, petrolatum oil, polydecenes, hydrogenated polyisobutene such as Parleam®.

According to a preferred embodiment of the invention, the fat(s) iv) are linear or branched hydrocarbon oils, which are volatile particularly selected from undecane, dodecane, isododecane, tridecane, and their mixtures of different oils, preferably volatile comprising isododecane in the mixture, or a mixture of undecane and tridecane, preferably a mixture of undecane and tridecane.

Advantageously, the composition comprises one or more fats, particularly liquid at 25° C. and at atmospheric pressure, preferably one or more oils, of the fatty medium, at a content ranging from 0.01% to 99.9% by weight, with respect to the total weight of the composition, preferably ranging from 0.1% to 70% by weight, preferably ranging from 1% to 50% by weight, preferably ranging from 5% to 20% by weight.

### v) Additional Surfactants

Preferably, the composition according to the invention comprises one or more surfactants v) different from iii), preferably non-ionic, from (poly)alkoxylated, preferably (poly)glycerolated, fatty alcohols.

The (poly)alkoxylated fatty alcohols suitable for the implementation of the invention are more specifically selected from alcohols including from 8 to 40 carbon atoms, preferably from 8 to 30 carbon atoms, and more specifically from 12 to 22 carbon atoms. The (poly)alkoxylated fatty alcohol(s) are preferably according to the following formula (V):

[Chem 7]

R^{a}—[O-ALK]ₙ—OH (V)

Formula (V) wherein:
R^{a} represents a C₈-C₃₀ preferably C₈-C₃₀ linear or branched alkyl or linear or branched alkenyl group optionally substituted by one or more hydroxyl groups; and
ALK represents a linear or branched (C₁-C₆)alkylene group, optionally substituted by one or more hydroxyl groups, preferably (C₁-C₄)alkylene, such as ethylene;
n represents an integer between 1 and 200 inclusive, preferably between 2 and 50 inclusive, more specifically between 8 and 30 inclusive such as 20.

According to a specific embodiment, the compounds having formula (V) are selected from mono or polyglycerolates, preferably polyglycerolates, they preferably include on average from 1 to 40 glycerol groups, more specifically from 10 to 30 glycerol groups such as 20. According to a specific embodiment of the invention, the surfactants v) are monoglycerolated or polyglycerolated preferably selected from the compounds having the following formulas:

[Chem 8]

RO[CH₂CH(CH₂OH)O]ₘH (Va),

RO[CH₂CH(OH)CH₂O]ₘH (Vb) or

RO[CH(CH₂OH)CH₂O]ₘH (Vc);

Formulas (Va) to (Vc) wherein:
R represents a linear or branched, saturated or unsaturated hydrocarbon radical, including from 8 to 40 carbon atoms and preferably from 10 to 30 carbon atoms;
m is a number between 1 and 30, preferably between 1 and 10, more specifically from 1.5 to 6. R can optionally comprise heteroatoms such as for example oxygen and nitrogen. In particular, R can optionally comprise one or more hydroxyl and/or ether and/or amide groups. R preferably denotes C₁₀-C₂₀ alkyl and/or alkenyl radicals, optionally mono or polyhydroxylated. More preferably, the surfactants v) are polyglycerolated and selected from Polyglyceryl-3 diisostearate and Polyglyceryl-4 isostearate, and mixtures According to a further embodiment, the compound(s) having formula (IV) are selected from fatty alcohols including from 8 to 22 carbon atoms and oxy(C₁-C₆)alkylenated by 1 to 30 moles of alkylene oxide.

Preferably, the additional surfactant(s) v) are selected from (poly)ethoxylated fatty alcohols having the following formula:

[Chem 9]

R^{a}—[O—CH₂—CH₂]ₙ—OH (V′)

Formula (V′) wherein:
R^{a} represents a C₈-C₄₀ preferably C₈-C₃₀ linear or branched alkyl or linear or branched alkenyl group optionally substituted by one or more hydroxyl groups; and
n represents an integer between 1 and 200 inclusive, preferably between 2 and 50 inclusive, more specifically between 8 and 30 inclusive such as 20.

More specifically, the surfactant(s) v) are selected from (poly)ethoxylated fatty alcohols; more specifically, fatty alcohols including from 8 to 22 carbon atoms and oxyethylenated by 1 to 30 moles of ethylene oxide (1 to 30 EO). Of these, mention can more specifically be made of lauric acid 2 EO, lauric alcohol 3 EO, decyl alcohol 3 EO, decyl alcohol 5 EO and oleic alcohol 20 EO.

A mixture of at least 2 different (poly)alkoxylated fatty alcohols can also be used, preferably of at least 2 different (poly)glycerolated fatty alcohols more preferably selected from the mixture of polyglyceryl-3 diisostearate and Polyglyceryl-4 isostearate.

### Water

The composition comprises an aqueous phase, i.e., also comprises water.

Water can form the continuous or dispersed phase.

The composition preferably comprises from 1% to 98% by weight of water with respect to the total weight of the composition, preferably from 10% to 80% by weight, preferably from 15% to 65% by weight.

According to an embodiment, the composition according to the invention comprises a greater quantity of fat than the quantity by weight of water.

According to the further embodiment of the invention, the composition comprises a greater quantity of water than the quantity of fat.

### Dosage Forms

The compositions according to the invention can be in different dosage forms, such as aqueous gels, direct (O/W) or invert (W/O) emulsions, multiple emulsions (O/W/O or W/O/W) or dispersions, where the aqueous phase is the continuous or dispersed phase.

The compositions according to the invention can be in the form of a dermatological or skin care composition, or in the form of a sun protection composition. It is then present in a non-colored form, optionally containing cosmetic or dermatological active ingredients different from compounds i) to iii). They can then be used as a care base for the skin or lips.

The composition according to the invention can also be in the form of a colored skin makeup product in particular a foundation, a blush, a blusher or eye shadow, or lip makeup such as a lipstick or eyelash or eyebrow makeup such as a mascara.

The composition according to the invention can also be in the form of an optionally colored hair product, in particular a conditioner, a care cream, or in the form of a deodorant.

The compositions according to the invention can be more or less fluid and have the appearance of a white or colored cream, an ointment, a milk, a lotion, a serum, a paste, a foam. They can optionally be applied on the skin in the form of a spray. They can also be in solid form, and for example in the form of a stick or a compact powder.

### Additional Ingredients

The compositions according to the invention can further comprise one or more additional ingredients selected from: a) gelling agents; b) optionally film-forming polymers; c) optionally thickening polymers; d) dyestuffs such as pigments, natural or synthetic direct dyes; e) fillers; f) UV filters; g) surfactants different from iii); h) natural or synthetic cosmetic active ingredients such as plant extracts; i) perfumes; j) as well as the salts thereof or the mixtures thereof; preferably d) one or more pigments; more preferably the pigment(s) of the invention are selected from carbon black, titanium oxides and iron oxides particularly black and micas coated with iron oxide, even more preferably iron oxides and titanium oxide.

The invention is now illustrated by the following example.

Unless specified otherwise, the quantities are expressed as a percentage by weight with respect to the total weight of the composition (% w/w).

Colorimetric evaluation Results in the L*a*b*system for evaluating the coloring of keratin material especially skin:

The color of the keratin material was evaluated in the L*a*b* system (by means for example of a MINOLTA*CM 3600D spectrocolorimeter (Illuminant D65)).

In this L*a*b* system, L* represents the lightness, a* indicates the green/red color axis and b* the blue/yellow color axis. The higher the value of L, the lighter or weaker the color. Conversely, the lower the value of L, the darker or much stronger the color. The higher the value of a*, the redder the shade, and the higher the value of b*, the more yellow the shade.

The variation in coloring between the untreated and treated keratin material is measured by (DE) according to the following equation:

$$Δ E = \sqrt{{( {L}^{*} - {L}_{0}^{*} )}^{2} + {( {a}^{*} - {a}_{0}^{*} )}^{2} + {( {b}^{*} - {b}_{0}^{*} )}^{2}}$$

In this equation, L*, a* and b* represent the values after treatment, and L₀*, a₀* and b₀* represent the values measured before treatment.

The greater the value of ΔE, the greater the difference in color between the colored keratin material and the uncolored keratin material. It is also possible to evaluate de color in the time of the treated material at T° and T later. In the latter case, L*, a* and b* of the equation represent the values after time T, and L₀*, a₀* and b₀* represent the values measured at the time T°.

Another criteria which can be taken into account is the chromaticity C* calculated from the equation C*=(a*²+b*²)^{1/2} wherein a* and b* are as defined herein before.

### EXAMPLE: FOUNDATION

### Description of Antimicrobial Efficacy Test Protocol

The efficacy of mixture i) and ii) and optionally iii) according to the invention is evaluated by means of the challenge test which is used to determine the level of antimicrobial protection of a composition.

It consists of an artificial contamination of the product under test by various microorganisms (bacteria, yeasts and molds) and monitoring of the number of viable microorganisms over time.

A product protected satisfactorily should enable decontamination of the microorganisms introduced that is more or less rapid according to the microbial strains, the type of product and the packaging material.

The product is distributed in the same number of pill bottles as microorganisms under test.

A calibrated microorganism suspension is introduced into each of these pill bottles.

The efficacy of the protection of the formulas is tested on a limited microbial spectrum, but selected from the species capable of contaminating the product, both in manufacture and during the use thereof. It comprises in this case one bacteria (Enterococcus faecalis), one yeast (Candida albicans) and one mold (Aspergillus brasiliensis).

The sampling performed to enumerate the microorganisms remaining in the product takes place after 7 days of contact between the product and the microorganism.

The dilutions performed to carry out the enumerations are seeded on Petri dishes.

The kinetics (growth or decline) for a given microorganism at a given time is then expressed as a log reduction.

The acceptability criteria are those defined in Annex B (Evaluation criteria for the preservation efficacy test) of the standard ISO11930:2019 (Cosmetics—Microbiology—Evaluation of the antimicrobial protection of a cosmetic product), reproduced hereinafter.

### Evaluation Criteria of the Standard ISO11930:2019

TABLE B.1
Evaluation criteria
Log reduction rate (Rₓ = lg N₀ − lg Nₓ) required^{a}

| | Microorganisms | | | | | | | |
|---|---|---|---|---|---|---|---|---|
| | Bacteria | | | C. albicans | | | A. brasiliensis | |
| | Sampling time | | | | | | | |
| | T7 | T14 | T28 | T7 | T14 | T28 | T14 | T28 |
| Crite- | ≥3 | ≥3 | ≥3 | ≥1 | ≥1 | ≥1 | ≥0^{c} | ≥1 |
| ria A | | and | and | | and | and | | |
| | | NI^{b} | NI | | NI | NI | | |
| Crite- | Not | ≥3 | ≥3 | Not | ≥1 | ≥1 | ≥0 | ≥0 |
| ria B | carried | | and | carried | | and | | and |
| | out | | NI | out | | NI | | NI |

^{a}In this test, a range of deviation of 0.5 log is acceptable (see 5.7).
^{b}NI: No increase in the number of microorganisms with respect to the preceding contact time.
^{c}Rₓ = 0 when lgN₀ = lgNₓ (no increase with respect to the initial number).

If the formulation meets criteria A, the microbiological risk is considered to be acceptable (the cosmetic product is protected against a microbial proliferation capable of involving a potential riskforthe user), and the cosmetic product meets the requirements of the Standard ISO 11930:2019 without any additional justification.

For the efficacy evaluation, only the criteria A “T7” will be taken into consideration.

For the mold Aspergillus brasiliensis, the challenge test result obtained will be deemed conforming if the required criterion after 28 days of product/microorganism contact is met from 7 days of contact.

### Evaluation of Protection Level of a Foundation

Compounds i), ii) and iii) are formulated in a foundation, a typical example of which has the following composition:

Composition 1 is according to the invention, whereas compositions 2 and 3 are comparative.

These compositions are prepared according to a conventional method for a person skilled in the art.

TABLE 2

| | | Composition | Composition |
|---|---|---|---|
| Ingredients | Composition | 2 | 3 |
| (% w/w) | 1 Invention | Comparative | Comparative |
| Vanillin i) | 0.25 | 0 | 0.25 |
| Tetrasodium | 0.2 | 0 | 0 |
| glutamate diacetate | | | |
| (Dissolvine GL-47-S | | | |
| from Akzo Nobel) ii) | | | |
| Capryloyl glycine iii) | 0.5 | 0.5 | 0 |
| Magnesium sulfate | 0.7 | 0.7 | 0.7 |
| Polyglyceryl-3 | 1 | 1 | 1 |
| diisostearate | | | |
| (surfactant v) | | | |
| Glycerol iii) | 5 | 5 | 5 |
| Polyglyceryl-4 | 1 | 1 | 1 |
| isostearate (surfactant | | | |
| v) | | | |
| Undecane & | 11 | 11 | 11 |
| Tridecane (fat volatile | | | |
| oil iv) | | | |
| (Cetiol UT) | | | |
| Synthetic | 1.6 | 1.6 | 1.6 |
| fluorphlogopite | | | |
| SYNAFIL S 1050 | | | |
| Titanium dioxide | 12.4 | 12.4 | 12.4 |
| Iron oxides | 2.44 | 2.44 | 2.44 |
| Water | Qs 100 | Qs 100 | Qs 100 |

The efficacy of each composition 1 to 4 is evaluated by means ofthe challenge test which is used to determine the level of antimicrobial protection of a composition.

The results are detailed in the tables herein below.

### Action on a Bacteria

The decline kinetics, expressed as log reductions, on the microbial strain Enterococcus faecalis after 7 days of microorganism/product contact are given in the table herein below:

TABLE 3

| Compositions | Log reductions after 7 days of contact |
|---|---|
| Composition 1 (invention) | <4 |
| Composition 2 (comparative) | <4 |
| Composition 3 (comparative) | 0.4 |

### Action on Yeast

The results on the microbial strain Candida albicans after 7 days of microorganism/product contact are given in the table herein below:

TABLE 4

| Compositions | Log reductions after 7 days of contact |
|---|---|
| Composition 1 (invention) | <4 |
| Composition 2 (comparative) | 0 |
| Composition 3 (comparative) | 2.5 |

### Action on Mold

The results on the microbial strain Aspergillus brasiliensis after 7 days of microorganism/product contact are given in the table herein below:

TABLE 5

| Compositions | Log reductions after 7 days of contact |
|---|---|
| Composition 1 (invention) | 3.3 |
| Composition 2 (comparative) | 0 |
| Composition 3 (comparative) | 4 |

The example shows that only the association according to the invention enables effective antimicrobial protection.

It is clear from the results of the above tables that the association of vanillin, capryloyl glycine and tetrasodium glutamate diacetate according to the invention has an excellent antimicrobial activity and provides a marked improvement of the level of microbiological protection, after 7 days of contact, in particular against the bacteria Enterococcus faecalis, the yeast Candida albicans and the mold Aspergillus brasiliensis.

For the same mixture, the level of decontamination observed on the 2 Enterococcus faecalis and Candida albicans strains meets criterion A described in the standard ISO 11930: 2019.

For the mold Aspergillus brasiliensis, the result is also considered to be conforming, as the required decontamination is achieved from 7 days of contact.

1. A composition comprising water and:
i) at least one (C₁-C₆)(alkyl)vanillin, one of the organic or mineral basic salts thereof, or one of the solvates thereof;
ii) at least one sequestering agent selected from the salts having the following formula (I), as well as the solvates thereof:
[⁻O—C(O)—CH₂]₂N—CH[C(O)O⁻]—(CH₂)₂—C(O)O⁻, nM⁺ (I)
Formula (I) wherein:
M⁺, identical or different, is selected from H⁺, alkali metal cations, alkaline earth metal cations and ammonium ions, and
n is an integer such that the number of M⁺ ensures the electroneutrality of the molecule, 4
and ethylene diamine disuccinic acid and the salts thereof, and
iii) optionally at least one additional antimicrobial, surfactants and mixtures thereof.

2. The composition according to claim 1, wherein i) (C₁-C₆)(alkyl)vanillin has the following formula (II): 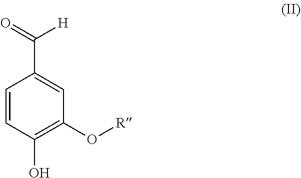
Formula (II) wherein R″ represents a linear or branched (C₁-C₆)alkyl group.

3. The composition according to claim 1, comprising a quantity of (C₁-C₆)(alkyl)vanillin, the organic or mineral basic salts thereof, or solvates thereof, between 0.01% and 2% by weight with respect to the total weight of the composition.

4. The composition according to claim 1, wherein ii) the sequestering agent having formula (I) is such that when M⁺, identical or different, is selected from alkali metal cations and ammonium ions, then n is equal to 4; and when M⁺, identical or different, is selected from alkaline earth metal cations, then n is equal to 2.

5. The composition according to claim 1, wherein ii) the sequestering agent having formula (I) is tetrasodium glutamate diacetate.

6. The composition according to claim 1, comprising a quantity ii) of sequestering agent between 0.01% and 1% by weight with respect to the total weight of the composition.

7. The composition according to claim 1, comprising at least one additional antimicrobial iii) selected from polyols having from 2 to 10 carbon atoms.

8. The composition according to claim 1, comprising at least one additional microbial iii) selected from the compounds having formula (IV) and the organic or mineral, acid or basic salts thereof, the optical isomers thereof, and the solvates thereof:
R₁—C(O)—NH—CH(R₂)—C(O)—OR₃ (IV)
Formula (IV) wherein:
R₁ represents a (C₆-C₂₀)alkyl group;
R₂ represents a hydrogen atom or a (C₁-C₆)alkyl group optionally substituted by a guanidine group optionally substituted by one or more (C₁-C₄)alkyl groups; and
R₃ represents a hydrogen atom or a (C₁-C₆)alkyl group;
preferably, the compounds having formula (IV) are selected from laurylethyl arginate and capryloyl glycine.

9. The composition according to claim 8, comprising at least one non-ionic surfactant having formula (IV at a content ranging from 0.01% to 3% by weight with respect to the total weight of the composition.

10. The composition according to claim 1, comprising iv) at least one fat.

11. The composition according to claim 1, comprising one or more surfactants v) different from iii).

12. The composition according to claim 1, comprising v) one or more monoglycerolated or polyglycerolated surfactants selected from the compounds having the following formulas:
RO[CH₂CH(CH₂OH)O]ₘH (Va),
RO[CH₂CH(OH)CH₂₀]ₘH (Vb) or
RO[CH(CH₂OH)CH₂O]ₘH (Vc);
Formulas (Va) to (Vc) wherein:
R represents a linear or branched, saturated or unsaturated hydrocarbon radical, including from 8 to 40 carbon atoms;
m is a number between 1 and 30;
R can optionally comprise heteroatoms.

13. The composition according to claim 10, comprising a greater quantity by weight of water than the quantity of fat.

14. The composition according to claim 1 further comprising one or more additional ingredients selected from:
a) gelling agents; b) optionally film-forming polymers; c) optionally thickening polymers; d) dyestuffs such as pigments, natural or synthetic direct dyes; e) fillers; f) UV filters; g) surfactants different from iii); h) natural and synthetic cosmetic active ingredients such as plant extracts;
i) perfumes; j) as well as the salts thereof or the mixtures thereof.

15. The composition according to claim 1, which has a pH between 3 and 10.

16. A non-therapeutic cosmetic treatment method of keratin material, comprising the application of a cosmetic composition according to claim 1 onto said keratin material.

17. A method for preserving a composition C1 comprising a physiologically acceptable medium, which comprises the incorporation of a composition according claim 1 15 in said composition C1.

18. A method for the microbial protection of a cosmetic composition against bacterial strains and/or molds, which comprises including in the cosmetic c composition a composition according to claim 1 as an antimicrobial agent.

19. The composition according to claim 2, comprising a quantity of (C₁-C₆)(alkyl)vanillin, the organic or mineral basic salts thereof, or solvates thereof, between 0.01% and 2% by weight with respect to the total weight of the composition.

20. The composition according to claim 2, wherein ii) the sequestering agent having formula (I) is such that when M⁺, identical or different, is selected from alkali metal cations and ammonium ions, then n is equal to 4; and when M⁺, identical or different, is selected from alkaline earth metal cations, then n is equal to 2.
